(19) United States
(12) Patent Application Publication
(10) Pub. No.: US 2025/0205553 A1
(43) Pub. Date: Jun. 26, 2025

(54) Player Information Exchange (P.I.E.) Protocol

(71) Applicant: Feng Xiao, Weston, FL (US)

(72) Inventor: Feng Xiao, Weston, FL (US)

(21) Appl. No.: 18/396,026

(22) Filed: Dec. 26, 2023

(51) Int. Cl. A63B 24/00

(52) U.S. Cl. CPC A63B 24/0062; A63B 2024/0068

(57) ABSTRACT

An internet-based player information and match results exchange standard protocol to allow different server systems and mobile applications communicate with each other using QR code to allow easy and convenient match information input and match result gathering for pickleball and other sports.

### BACKGROUND OF THE INVENTION

Pickleball is the fastest growing sport in America. There are estimated more than 20 million players now in America. Many players play more than five days each week and usually play for multiple hours on the days that they play.

Typically, players go to the same facility and play with a rather stable group of players for normal daily games. Players want to track their game results and see the history of their rankings. However, these daily games are usually not tracked because it takes too much time to track a game score.

At the same time, there are many websites and systems that store their registered players' information. Foreseeably, there will be many different mobile applications that handle game scores and play history tracking.

This Player Information Exchange protocol can standardize the information exchange, simplify the game setup and score keeping for all pickleball players, websites, systems and mobile apps.

I found no prior art when I conducted a general search on USPTO for Pickleball Player and Game Information Exchange Standard.

### SUMMARY OF THE DISCLOSURE

The primary objective of this invention is to provide a standard for player and match information exchange for pickleball. The standard must be easy to implement on the server side and mobile app side, quick to operate for all games, and extendable for more sophisticated functions.

My invention fulfills the objectives by defining an XML based information exchange standard that can be easily implemented by websites, systems, and mobile applications written in any modern programming language. By encoding the P.I.E. adaptor url and player ID in a QR code, during any game play, players can quickly setup a game by scanning the QR code, which takes a matter of seconds. Lastly, the standard can be further extended for other sports such as tennis, ping pong, badminton, golf.

### BRIEF DESCRIPTION OF THE DRAWINGS

FIG. 1 is a logical illustration of when a user registers to a PIE compliant website, the web server generates a unique ID for the user and renders a QR code for the user. The QR code can then be stored on the user's mobile phone or printed and pasted to the user's paddle.

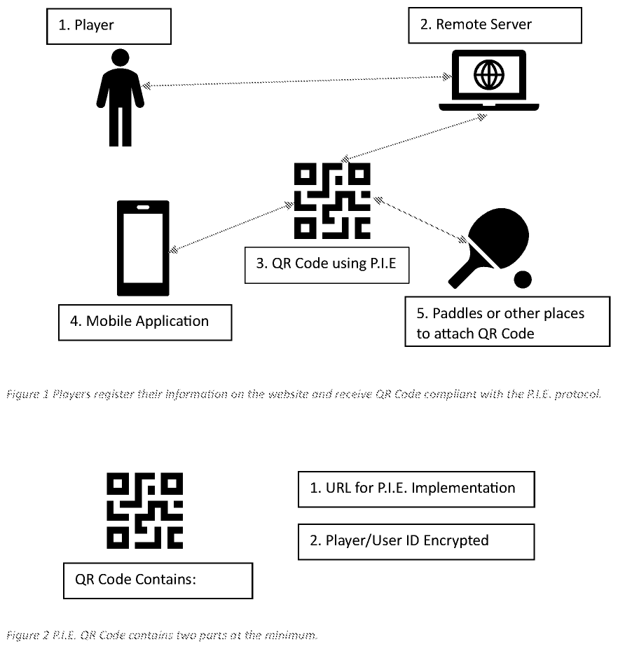

FIG. 2 illustrates what the QR Code must contain at the minimum. It must have the PIE compliant adaptor implementation url, for example, http://www.funtimepb.com/PIEadaptor.asmx, and the user's unique ID in an encrypted format. The implemented adaptor must be capable of decoding the encrypted user ID and finding the corresponding user's information.

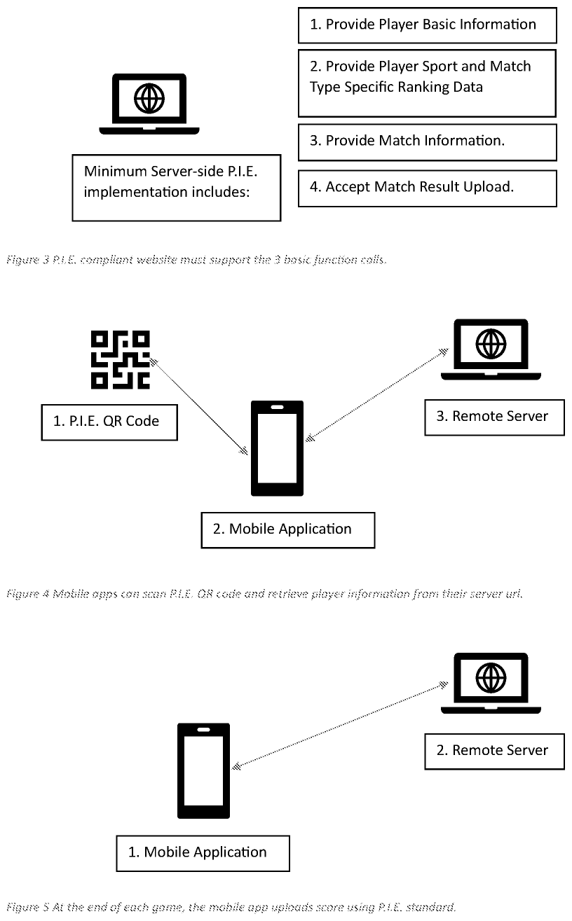

FIG. 3 shows that the PIE adaptor must implement at the minimum the following functions:
1. GetUserBasicInfo function that takes the encrypted user ID as input variable and returns the user's information.
2. GetUserRankingInfo function that takes the name of the sport, match type, and user ID as input variable and returns user rankings.
3. GetMatchInfo function that takes a match ID as input variable and returns all players information for the match.
4. SetMatchResult function that takes a match ID and score for each player as input variable. It will update player match score history and ranking accordingly.

FIG. 4 shows the mobile application can scan the QR code for player or match and then retrieve the player or match information from the encoded url, which will accept the input and return the requested information following the PIE standard. The mobile application then uses that information to display match or player information as needed.

FIG. 5 shows at the end of the game, the mobile application updates the score in its local storage and then uploads to the PIE compliant server it stored at the beginning of the match. The server then processes the match score for each player accordingly.

### DETAILED DESCRIPTION OF THE INVENTION

1. Server-side components are implemented using webservices and can be discovered using WSDL.
a. A PIE compliant server can take any user registration and generate a QR code that includes the PIE adaptor implementation url, and the user's encrypted ID. For example, a user's QR code from www.funtimepb.com could be http://www.funtimepb.com/pieadaptor.asmx?UID=ABCDE.
b. When the GetUserBasicInfo function is called, the adaptor takes encrypted UID as input and returns the following XML block:

<BasicInfo PIEVersion=”1.0”>
<FirstName>***</FirstName>
<LastName>***</LastName>
<Gender>M or F</Gender>
<YOB>****</YOB>
</BasicInfo>
For example, https://www.funtimepb.com/pieadaptor.asmx could
return the following sample user information:
<BasicInfo PIEVersion=”1.0”>
<FirstName>Feng</FirstName>
<LastName>Xiao</LastName>
<Gender>M</Gender>
<YOB>1973</YOB>
</BasicInfo>

c. When the GetUserRankingInfo function is called, the adaptor takes encrypted UID, Sports Name, and Match Type as input. The input XML are defined as:

<RankingRequest PIEVersion=”1.0”>
<Sport>Pickleball</Sport>
<MatchType>MD</MatchType>
<UID>ABCDEF</UID>
</RankingRequest>
The function returns the following XML block:
<RankingInfo PIEVersion=”1.0”>
<Sport>****</Sport>
<MatchType>**</MatchType>
<Ranking>
<Rank Name=*** Type=**>####</Rank>
<Rank Name=*** Type=**>####</Rank>
</Ranking>
</RankingInfo>
For example, https://www.funtimepb.com/pieadaptor.asmx could
return the following ranking information:
<RankingInfo PIEVersion=”1.0”>
<Sport>Pickleball</Sport>
<Match>MD(Mixed Double)</Match>
<Ranking>
<Rank Name=DUPR Type=C>4.58</Rank>
<Rank Name=PIE Type=S>450</Rank>
</Ranking>
</RankingInfo>

d. When the GetMatchInfo function is called, the adaptor takes encrypted Match ID as input. The function returns the following XML block:

<MatchInfo PIEVersion=”1.0”>
<Sport>****</Sport>
<EventName>****</ EventName >
<MatchName>****</MatchName>
<MatchDate>****</MatchDate>
<Facility>*****</Facility>
<MatchType>****</MatchType>
<RankingSetup>****</ RankingSetup>¹
<Players>
<Player Position=”A1”>
<FirstName>***</FirstName>
<LastName>***</LastName>
<Gender>M or F</Gender>
<YOB>****</YOB>
<Rank>####</Rank>
</Player>
<Player Position=”A2”>
<FirstName>***</FirstName>
<LastName>***</LastName>
<Gender>M or F</Gender>
<YOB>****</YOB>
<Rank>####</Rank>
</Player>
<Player Position=”B1”>
<FirstName>***</FirstName>
<LastName>***</LastName>
<Gender>M or F</Gender>
<YOB>****</YOB>
<Rank>####</Rank>
</Player>
<Player Position=”B1”>
<FirstName>***</FirstName>
<LastName>***</LastName>
<Gender>M or F</Gender>
<YOB>****</YOB>
<Rank>####</Rank>
</Player>
</Players>
</MatchInfo>
¹This references another USPTO application about weighted ranking adjustment algorithm and implementation by the same inventor.

e. When the SetMatchResult function is called, the adaptor takes encrypted Match ID and Scores as input. The input XML are defined as:

<MatchResult PIEVersion=”1.0”>
<MatchID>###</MatchID>
<Scores>
<Player Position=”A1” UID=”****” Score=”###”></Player>
<Player Position=”A2” UID=”****” Score=”###”></Player>
<Player Position=”B1” UID=”****” Score=”###”></Player>
<Player Position=”B2” UID=”****” Score=”###”></Player>
</Scores>
</MatchResult>

The function would update all player's match history and adjust their ranking accordingly. It also returns the following XML block:

<MatchResultResponse PIEVersion=”1.0”>
<MatchID>###</MatchID>
<UpdateResponses>
<Player Position=”A1” UID=”****”>Yes</Player>
<Player Position=”A2” UID=”****”>No</Player>
<Player Position=”B1” UID=”****”>No</Player>
<Player Position=”B2” UID=”****”>Yes</Player>
</UpdateResponses>
</MatchResultResponse>

Basically, depending on whether this player is actually registered on the same website as the match provider, the update may or may not succeed. The feedback allows the mobile app to display information accordingly.

2. Mobile application will perform the following functions according to the PIE standard:
a. Scan the PIE Player QR Code, decode the information in the QR Code, and submit webservice calls to the embedded URL to retrieve individual player's basic information.
b. Do the same to retrieve the player's sport specific and match type dependent ranking information. For example, a person can be 4.0 in singles pickleball match and at the same time, 4.5 for mixed doubles. This also includes other sports such as tennis.
c. Scan the PIE Match QR Code and retrieve all match related information including players' name and ranking. Then, display such information on the screen. Allow the user to further configure the display such as which player is standing on which side of the court and keep track of the score as game is progressing. This is very useful for referees.² This also references another provisional USPTO application by the same inventor about an electronic device that helps tracking the score in a pickleball match.
d. At the end of the match, upload the scores to the server using the embedded url in the match QR code. Receive the response from server and display the information on screen accordingly.

It is to be understood that while a generic illustration of the invention is drawn above, it is not to be limited to specific form or arrangement of parts herein described and shown. It will be apparent to those skilled in the art that various changes may be made without departing from the scope of the invention and the invention is not to be considered limited to what is shown and described in the specification and drawings.

Having thus described my invention, I hereby claim the following:

1. A common data exchange protocol for different systems to transmit and retrieve player and match information for various sports, including but not limited to pickleball.

2. Using a QR code that can be stored on user's phone or printed as a sticker and attached to user's paddle to allow quick scanning and setting up spontaneous matches in a mobile app.

3. Using a QR code that is generated by a tournament system that allows the mobile application to scan the QR code to retrieve the match related information, and all player's information for that match.

4. Practical and convenient implementation that allows decoupling of the server implementation and the mobile application development. As long as they both support the PIE protocol, they can work with each other.
